(19) United States
(12) Patent Application Publication
(10) Pub. No.: US 2011/0071363 A1
(43) Pub. Date: Mar. 24, 2011

(54) SYSTEM AND METHOD FOR USING PREDICTIVE MODELS TO DETERMINE LEVELS OF HEALTHCARE INTERVENTIONS

(75) Inventors: Michael Montijo, Nashville, TN (US); Jason Grant, Brentwood, TN (US); John Turner, Nashville, TN (US); Adam Hobgood, Smyrna, TN (US)

(73) Assignee: Healthways, Inc., Franklin, TN (US)

(21) Appl. No.: 12/616,754

(22) Filed: Nov. 11, 2009

Related U.S. Application Data
(60) Provisional application No. 61/244,865, filed on Sep. 22, 2009.

(51) Int. Cl. A61B 5/00; G06Q 10/00; G06Q 50/00; G06N 3/02; G06N 5/02

(52) U.S. Cl. 600/300; 705/2; 706/21; 706/52

(57) ABSTRACT

A computer implemented method may be used for targeting patients within a population for increased medical interventions. In one embodiment, predictive models are used to determine whether a patient is likely to be near the end of life, whether the patient's healthcare costs are stabilizing, whether the patient is likely to have high future healthcare costs, and whether the patient is likely to have high future clinical risk. In one embodiment, scores from these predictive models are used to determine the level of medical intervention for a particular patient.

### CROSS-REFERENCE TO RELATED APPLICATIONS

This application claims the benefit of U.S. Patent Application No. 61/244,865 filed on Sep. 22, 2009, which is hereby incorporated herein by reference in its entirety. This application is related to U.S. patent application Ser. No. 12/340,491 filed on Dec. 19, 2008, which is hereby incorporated herein by reference in its entirety.

### BACKGROUND OF THE INVENTION

1. Field of the Invention

This application relates to the use of multiple predictive models to identify a sub-group of patients to receive a particular level of medical care, according to predefined criteria.

2. Description of the Related Art

Because patients in a healthcare management system have varying health conditions, it is desirable to provide a level of medical care that varies with each patient's medical history rather than providing the same type of care to all patients. Varying the level of medical care decreases the overall cost of providing healthcare because increased healthcare services and cost can be targeted to the patients more likely to benefit from it.

### SUMMARY OF THE INVENTION

In one embodiment, a computerized method for targeting a group of patients for medical care, the method comprises receiving health information about a plurality of patients, selecting a subset of the plurality of patients, comparing the relative associated future healthcare costs and clinical risk of the subset of the plurality of patients, and assigning a level of medical intervention for each of the patients in the selected subset based on the comparison of the patients. The received health information may comprise healthcare insurance claims information. The method may further comprise creating a medical care plan based on the assigned level of medical intervention. Selecting a plurality of patients may comprise selecting a plurality of patients based on the likelihood of each patient dying within a particular time range. Selecting a plurality of patients may comprise selecting a plurality of patients based on the likelihood of the future healthcare costs for each patient stabilizing. Assigning a level of medical intervention may comprise assigning patients with high future healthcare costs and high clinical risk, to an increased level of medical intervention. The method may further comprise periodically reassigning patients to a different level of medical intervention. The method may further comprise assigning the non-selected patients to a level of medical intervention. A computing device may assign the level of medical intervention. The method may further comprise delivering the assigned level of medical intervention to a patient in the selected subset. Selecting the subset of the plurality of patients may comprise both selecting a plurality of patients based on the likelihood of each patient dying within a particular time range and selecting a plurality of patients based on the likelihood of the future healthcare costs for each patient stabilizing.

In one embodiment, a computerized method for targeting a group of patients for medical care, the method comprises receiving health information associated with each of a plurality of patients, receiving output from multiple predictive models about the plurality of patients, comparing a subset of the plurality of patients relative to one another based on the output of the multiple predictive models, and assigning a level of medical intervention for each of the patients based on the comparison of the patients. The received health information may comprise healthcare insurance claims information. The method may further comprise creating a medical care plan based on the assigned level of medical intervention. A computing device may assign the level of medical intervention. The output from the multiple predictive models may comprise output indicative of each patient's likelihood of dying in the near term. The output from the multiple predictive models may comprise output indicative of the likelihood of each patient's healthcare costs stabilizing. The output from the multiple predictive models may comprise output indicative of the relative level of each patient's future healthcare costs. The output from the multiple predictive models may comprise output indicative of each patient's relative future clinical risk. Patients with an associated predictive model output indicative of a higher relative level of future healthcare costs and a higher relative future clinical risk may be assigned to an increased level of medical intervention. The levels of medical intervention may comprise a surveillance level, an end of life level, and an intermediate level of medical intervention. The method may further comprise periodically reassigning patients to a different level of medical intervention. The method may further comprise delivering the assigned level of medical intervention to a patient based on the comparison of the patients. The output from the multiple predictive models may comprises two or more outputs selected from the group consisting of output indicative of each patient's likelihood of dying in the near term, output indicative of the likelihood of each patient's healthcare costs stabilizing, output indicative of the relative level of each patient's future healthcare costs and output indicative of each patient's relative future clinical risk. The output from the multiple predictive models may comprise output indicative of each patient's likelihood of dying in the near term, output indicative of the likelihood of each patient's healthcare costs stabilizing, output indicative of the relative level of each patient's future healthcare costs, and output indicative of each patient's relative future clinical risk.

In one embodiment, a computerized method for targeting a group of patients for medical care, the method comprises selecting a plurality of patients, determining an estimated medical factor associated with each patient, comparing the patients based on the associated medical factor of each patient, and assigning a level of medical intervention for each patient based on the relative estimated medical factor of each patient. The estimated medical factor may comprise the estimated future medical costs associated with each patient. The estimated medical factor may further comprise the estimated future clinical risk associated with each patient. Patients with a high estimated future medical costs and high estimated clinical risk relative to the other patients may be assigned to an increased level of medical intervention. Selecting a plurality of patients may comprise selecting a plurality of patients based on the likelihood of each patient dying within a particular time range. Selecting a plurality of patients may comprise selecting a plurality of patients based on the likelihood of the future medical costs for each patient stabilizing. Determining an estimated medical factor may comprise analyzing output from one or more predictive models. The one or more predictive models may be neural networks. The levels of medical intervention may comprise a surveillance level and active care level of medical intervention. The method may further comprise creating a medical plan based on the assigned level of medical intervention. The method may further comprise assigning the non-selected patients to a level of medical intervention. A computing device may assign the level of medical intervention. Selecting a plurality of patients may comprise both selecting a plurality of patients based on the likelihood of each patient dying within a particular time range and selecting a plurality of patients based on the likelihood of the future healthcare costs for each patient stabilizing. The method may further comprise delivering the assigned level of medical intervention to a patient based on the relative estimated medical factor of said patient.

In one embodiment, a computerized system for targeting a group of patients for medical care, the system comprises a memory to store health information about a plurality of patients and a processor to perform: selecting a subset of the plurality of patients, comparing the relative associated future healthcare costs and clinical risk of the subset of the plurality of patients, and assigning a level of medical intervention for each of the patients in the selected subset based on the comparison of the patients. The received health information may comprise healthcare insurance claims information. The processor may further perform creating a medical care plan based on the assigned level of medical intervention. Selecting a plurality of patients may comprise selecting a plurality of patients based on the likelihood of each patient dying within a particular time range. Selecting a plurality of patients may comprise selecting a plurality of patients based on the likelihood of the future healthcare costs for each patient stabilizing. Assigning a level of medical intervention may comprise assigning patients with high future healthcare costs and high clinical risk to an increased level of medical intervention. The processor may further perform periodically reassigning patients to a different level of medical intervention. The processor may further perform assigning the non-selected patients to a level of medical intervention. The processor may further perform delivering the assigned level of medical intervention to a patient in the selected subset. Selecting the subset of the plurality of patients may comprise both selecting a plurality of patients based on the likelihood of each patient dying within a particular time range and selecting a plurality of patients based on the likelihood of the future healthcare costs for each patient stabilizing.

In one embodiment, a computerized system for targeting a group of patients for medical care, the system comprises a memory for storing health information associated with each of a plurality of patients and a processor to perform: receiving output from multiple predictive models about the plurality of patients, comparing a subset of the plurality of patients relative to one another based on the output of the multiple predictive models, and assigning a level of medical intervention for each of the patients based on the comparison of the patients. The received health information may comprise healthcare insurance claims information. The processor may further perform creating a medical care plan based on the assigned level of medical intervention. The output from the multiple predictive models may comprise output indicative of each patient's likelihood of dying in the near term. The output from the multiple predictive models may comprise output indicative of the likelihood of each patient's healthcare costs stabilizing. The output from the multiple predictive models may comprise output indicative of the relative level of each patient's future healthcare costs. Patients with an associated predictive model output indicative of a higher relative level of future healthcare costs and a higher relative future clinical risk may be assigned to an increased level of medical intervention. The levels of medical intervention may comprise a surveillance level, an end of life level, and an intermediate level of medical intervention. The processor may further perform periodically reassigning patients to a different level of medical intervention. The output from the multiple predictive models may comprise two or more outputs selected from the group consisting of output indicative of each patient's likelihood of dying in the near term, output indicative of the likelihood of each patient's healthcare costs stabilizing, output indicative of the relative level of each patient's future healthcare costs and output indicative of each patient's relative future clinical risk. The output from the multiple predictive models may comprise output indicative of each patient's likelihood of dying in the near term, output indicative of the likelihood of each patient's healthcare costs stabilizing, output indicative of the relative level of each patient's future healthcare costs, and output indicative of each patient's relative future clinical risk.

In one embodiment, a computerized system for targeting a group of patients for medical care, the system comprises a processor to perform: selecting a plurality of patients, determining an estimated medical factor associated with each patient, comparing the patients based on the associated medical factor of each patient, and assigning a level of medical intervention for each patient based on the relative estimated medical factor of each patient. The estimated medical factor may comprise the estimated future medical costs associated with each patient. The estimated medical factor may further comprise the estimated future clinical risk associated with each patient. Patients with a high estimated future medical costs and high estimated clinical risk relative to the other patients may be assigned to an increased level of medical intervention. Selecting a plurality of patients may comprise selecting a plurality of patients based on the likelihood of each patient dying within a particular time range. Selecting a plurality of patients may comprise selecting a plurality of patients based on the likelihood of the future medical costs for each patient stabilizing. Determining an estimated medical factor may comprise analyzing output from one or more predictive models. The one or more predictive models may be neural networks. The levels of medical intervention may comprise a surveillance level and active care level of medical intervention. The processor may further perform creating a medical plan based on the assigned level of medical intervention. The processor may further perform assigning the non-selected patients to a level of medical intervention. Selecting a plurality of patients may comprise both selecting a plurality of patients based on the likelihood of each patient dying within a particular time range and selecting a plurality of patients based on the likelihood of the future healthcare costs for each patient stabilizing.

### BRIEF DESCRIPTION OF THE DRAWINGS

FIG. 1 is a block diagram illustrating one embodiment of a healthcare management system.

FIG. 2 is a block diagram illustrating one embodiment of plural predictive models that may be used in the healthcare management system shown in FIG. 1.

FIG. 3 is a flow chart illustrating one embodiment of a process for assigning a level of medical intervention to a plurality of patients.

FIG. 4 is a block diagram illustrating one embodiment of an example of a matrix used for assigning patients to a level of medical intervention.

FIG. 5 is a diagram of one embodiment of a chart depicting possible assignments of patients to levels of medical intervention.

FIG. 6 is a flow chart illustrating one embodiment of a process for assigning a patient to a level of medical intervention.

### DETAILED DESCRIPTION OF CERTAIN EMBODIMENTS

The system, method, and devices of the invention each have several aspects, no single one of which is solely responsible for its desirable attributes. Without limiting the scope of this invention as expressed by the claims which follow, its more prominent features will now be discussed briefly. After considering this discussion, and particularly after reading the section entitled “Detailed Description of Certain Embodiments” one will understand how the features of this invention provide advantages that include efficiently targeting patients for particular types of healthcare interventions.

The following detailed description of certain embodiments presents various descriptions of specific embodiments of the invention. However, the invention can be embodied in a multitude of different ways as defined and covered by the claims. In this description, reference is made to the drawings wherein like parts are designated with like numerals throughout.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

The system is comprised of various modules, tools, and applications as discussed in detail below. As can be appreciated by one of ordinary skill in the art, each of the modules may comprise various sub-routines, procedures, definitional statements and macros. In some embodiments, each of the modules are separately compiled and linked into a single executable program. Therefore, the following description of each of the modules is used for convenience to describe the functionality of the system. Thus, the processes that are undergone by each of the modules may be arbitrarily redistributed to one of the other modules, combined together in a single module, or made available in, for example, a shareable dynamic link library.

The system modules, tools, and applications may be written in any programming language. For example, in some embodiments, the applications may be written in C, C++, C#, BASIC, Visual Basic, Pascal, Ada, Java, HTML, XML, or FORTRAN, and executed on an operating system. In some embodiments, the operating system may be Windows, Macintosh, UNIX, Linux, VxWorks, or another variant of the foregoing operating system. C, C++, BASIC, Visual Basic, Pascal, Ada, Java, HTML, XML and FORTRAN are industry standard programming languages for which many commercial compilers can be used to create executable code.

Clinical risks, health costs, and patient needs vary among patients. In order to decrease costs and to provide better medical care, it is desirable to assign each patient to a particular type of medical intervention. The medical intervention may involve, for example, the type of contact with a patient, the frequency of contact with the patient, and the types of health and wellness programs offered. This may result in decreasing medical costs and increasing the wellness of the patients. In certain embodiments, with highly accurate targeting, a healthcare service provider may be able to focus interventions only on the select group of patients with the highest risk for a high cost event that may be due, for example, to health risk factors or gaps in care.

In one embodiment, a healthcare management system combines the scores from multiple predictive models in order to determine a level of medical intervention for each patient in a group of patients. The use of multiple predictive models allows the level of medical intervention to be based on the prediction of multiple types of factors, rather than a single type of factor, such as cost. The multiple factors can also allow the assignment of medical intervention to address multiple goals, such as reduction of cost, reduced risk, improved clinical metrics, or patient satisfaction. In one embodiment, the particular predictive models used can be varied based on the goals selected by the user. For example, in one embodiment an employer or healthcare plan can choose particular goals and the healthcare management system adjusts the predictive models accordingly. Because multiple factors can be addressed through the use of multiple predictive models, the use of multiple predictive models leads to more accurate and precise targeting of a sub-group. If only one predictive model is used, all of the factors may not be taken into account, and individuals may be missed or the results from the model may be over-inclusive.

In one embodiment, the healthcare management system combines scores from multiple predictive models so that individuals can be assigned to levels of medical intervention based on factors other than, or in addition to, disease. This is because targeting medical intervention based on disease has been found to not be the best way to prospectively identify those who were likely to drive high costs. Such a total population approach is desirable because it may identify risk and cost regardless of the existence or absence of chronic conditions.

A healthcare management system using multiple predictive models has a wide variety of applicability. For example, it could be used by a health insurance plan, Medicare, or the government. In one embodiment, the healthcare management is hosted by a particular entity which provides the output from the combination of the multiple predictive models. In another embodiment, the healthcare management system or individual components of it may be a usable product sold to customers. In one embodiment, the predictive model system is part of a larger networked healthcare management system. Certain embodiments of the system and method as described herein may be part of a product such as Embrace™, soon to be available from Healthways, Inc. In one embodiment, the healthcare management system may be tailored to a particular type of care, such as care for senior citizens.

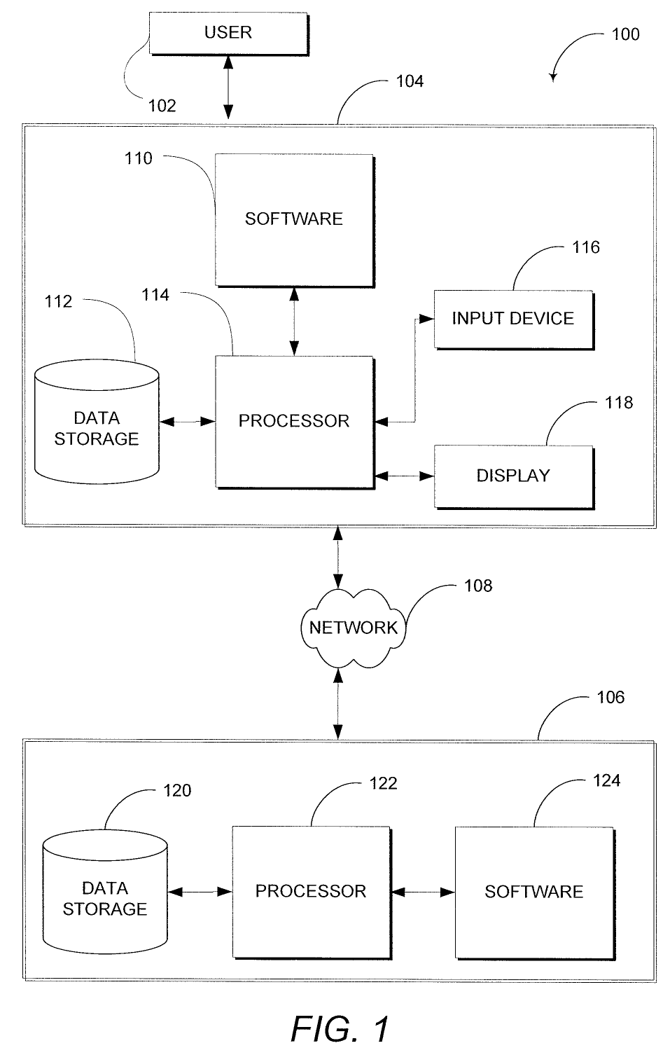

FIG. 1 is a system diagram illustrating one embodiment of a healthcare management system 100. In one embodiment, a mobile or fixed computing device 104 is operated by a user 102. The computing device 104 can be a handheld computing device or other portable computing device such as a Palm, Pocket personal computer (PC), Linux based handheld, PDA, smartphone, Tablet PC, or PC having a display. Alternatively, the computing device 104 may be a personal computer having a built in or separate display. The computing device 104 in certain embodiments operates in a stand-alone (independent) manner. In other embodiments, the computing device 104 is in communication with one or more computing devices 106, such as a server, via a network 108. The computing devices 106 include one or processors 122, data storage 120, and system software 124 executed by the processor(s) 122.

In certain embodiments, the data storage 120 stores one or more databases used by the system, and stores patient medical records. The processor(s) 122 are in communication with the database(s) via a database interface, such as structured query language (SQL) or open database connectivity (ODBC). In certain embodiments, the data storage 120 is not included in computing device 106, but is in data communication with the computing device 106 via the database interface.

The healthcare system 100 may include a network 108, which may represent a local area network (LAN), a wide area network (WAN), the Internet, or another connection service. The connection from the computing device 104 to the network 108 can be a wireless or a satellite connection or a wired or direct connection. In certain embodiments, the server(s) are part of a web site, such as on an intranet or the Internet.

The computing device 104 includes a processor 114, an integral or separate display 118, and one or more input devices 116. The processor 114 is in data communication with a data storage 112 for storing one or more databases having data such as medical data used by the system. In certain embodiments, the data storage 112 stores data such as patient medical records. System software 110 is executed by the processor 114. In certain embodiments, the system software 110 includes an application graphical user interface (GUI). The application GUI can include a database interface to the data storage 112 of the computing device. In certain embodiments, the software is loaded from the data storage 112. In embodiments where the computing device 104 communicates with a web site, the processor utilizes browser software in place of or in addition to the software 110. The network 108 may connect to a user computer 104, for example, by use of a modem or by use of a network interface card. A user 102 at computer 104 may utilize a browser to remotely access the programs using a keyboard and/or pointing device and a visual display, such as monitor. Alternatively, the browser is not utilized when the programs are executed in a local mode on computer 104. A video camera may be optionally connected to the computer 104 to provide visual input.

In one embodiment, the processes discussed herein are executed on the processor 114. In another embodiment, the processes are executed on the processor 122 with output sent via the network 108 to the computing device 104. In one embodiment, the programs and databases reside on a group of servers that are interconnected by a LAN and a gateway to a network. Alternatively, the programs and databases reside on a single server that utilizes network interface hardware and software.

Various other devices may be used to communicate with the computing device 106. If the servers are equipped with voice recognition or DTMF hardware, the user can communicate with the program by use of a telephone. Other connection devices for communicating with the computing device 106 include a portable personal computer with a modem or wireless connection interface, a wireless device such as a mobile telephone or a smart phone, a cable interface device connected to a visual display, or a satellite dish connected to a satellite receiver and a television. Other ways of allowing communication between the user 102 and the servers 106 are envisioned.

As used herein, actions performed by the healthcare management system 100 and/or processes described herein may be performed by any suitable hardware or software, including the processor 114 or the processor 122.

Any suitable predictive models may be used to assign a level of medical care to a group of patients. The actual predictive models used may likely differ depending on a program offering. The concept of using multiple predictive models can be adapted to different types of healthcare. For instance, a program offering could be a management program offered for care to senior citizens. In another embodiment, an offering could be a management program offered to patients with a terminal diagnosis. In another embodiment, the program could offer care to a sub group with the greatest amount of risk for a particular illness. In another embodiment, the group of members that is most likely to experience increased health costs is targeted from the entire population. In another embodiment, a predictive model is used to predict actionable risks from healthcare claims, such as insurance claims. Another predictive model may be used predicts risks that can be mitigated based on health risk assessment data.

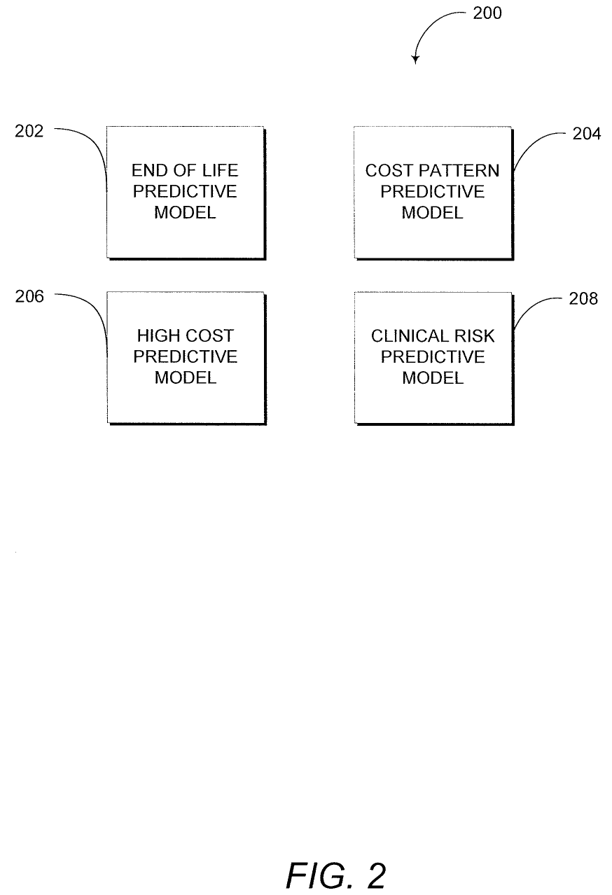

FIG. 2 is a block diagram illustrating one embodiment of predictive models 200 that may be used in the healthcare management system 100. In one embodiment, the predictive models include an end of life predictive model 202, a cost pattern predictive model 204, a high cost predictive model 206, and a clinical risk predictive model 208. The healthcare management system 100 may use output from one or more of these models to assign a level of medical intervention to a patient. In one embodiment, separate types of inputs may be used in each of the predictive models.

The end of life predictive model 202 may be a neural network model for predicting the probability of the patient's death in the near term. In one embodiment, the end of life predictive model 202 uses methods described in U.S. patent application Ser. No. 12/340,491, which is hereby incorporated herein by reference in its entirety. In one embodiment, the end of life predictive model 202 outputs a score indicative of the probability that a patient will die within a predetermined time period. In another embodiment, the end of life predictive model 202 outputs a comparative ranking of a group of patients in order of their increasing or decreasing likelihood to die within a predetermined time period. In one embodiment, the end of life predictive model 202 receives input used to tailor the model in a certain manner. In one embodiment, the predictive models 200 are neural networks.

The cost pattern predictive model 204 may be a neural network for determining the pattern of a patient's claims cost. In traditional disease management, members with accelerating and de-accelerating medical costs are equally targeted with routing intervention, resulting in induced care and increased cost for those who would naturally have regressed to the mean level of claims cost. In one embodiment, the cost pattern predictive model 204 may output a score for each patient indicative of whether the patient's medical costs are stabilizing. In another embodiment, the cost pattern predictive model 204 outputs an index for each patient used to rank the patients based on their relative health care cost stability.

The high cost predictive model 206 may be a neural network for predicting future high claim costs. In one embodiment, the high cost predictive model 206 determines an estimated dollar amount of a patient's future medical costs over a particular time period. In another embodiment, the high cost predictive model 206 assigns an index value to each patient representative of the patient's estimated future medical costs compared to other patients. In yet another embodiment, the future high cost predictive model 206 ranks the patients in order of increasing or decreasing estimated future medical costs.

The clinical risk predictive model 208 may be a predictive model used to determine a level of clinical risk. In one embodiment, the clinical risk predictive model 208 outputs an adjusted risk index indicative of the relative clinical risk of the particular patient. The clinical risk may be representative of the patient's health risk which may be determined by factors such as the patient's diseases, overall medical condition, or age. In one embodiment, the clinical risk predictive model 208 may be altered to receive different types of factors as inputs.

In one embodiment, the healthcare management system 100 executes one or more predictive models. In another embodiment, the healthcare management system 100 receives output from one or more predictive models and processes the output from the one or more predictive models. For example, the predictive model scores may be received from an outside entity. In one embodiment, one or more of the predictive models are run by the Centers for Health Research (“CHR”). In one embodiment, D2 Hawkeye, available from Verisk Health, 130 Turner Street, Seventh Floor, Waltham, Mass. 02453, calculates an adjusted clinical risk score based on its execution of the clinical risk predictive model 208.

In one embodiment, the healthcare management system 100 aggregates healthcare information, such as healthcare claim data. The healthcare information may then be used as a factor set for the predictive models. In one embodiment, the healthcare management system 100 uses the factor set and executes the predictive models. In another embodiment, after the processor 101 aggregates healthcare data, the aggregated healthcare information may be supplied to an outside entity, which uses the healthcare information to create and execute the predictive models. The healthcare management system 100 may format the healthcare information into a particular format prior to the healthcare information being sent to the outside entity. In one embodiment, the healthcare management system 100 may alter the factor set based on the type of predictive information desired. In one embodiment, the factor set for the predictive models is supplied by an outside entity. In one embodiment, the factors are created by analyzing healthcare medical claims data from multiple individuals and combined into one variable for each patient population. The factors may then be stored in a variable log.

In one embodiment, the predictive model scores are stored in the data storage 120 and accessed by the healthcare management system 100. In another embodiment, the predictive model scores are stored remotely from the healthcare management system 100. In one embodiment, the scores for each of the predictive models are stored independently of any factors or inputs used to create the scores. This allows for the predictive model scores to be stored in a relational database in which each score is stored on a table row without the need to develop any new columns for the table. This type of storage may be more scalable for a large organization that is likely to receive a large number of future predictive model scores.

In one embodiment, the healthcare management system 100 post processes the predictive model scores received from the predictive models. In some embodiments, post scoring is started by querying the table used to hold the names of post score procedures used in the predictive models. In one embodiment, the post scoring is done on a regular basis.

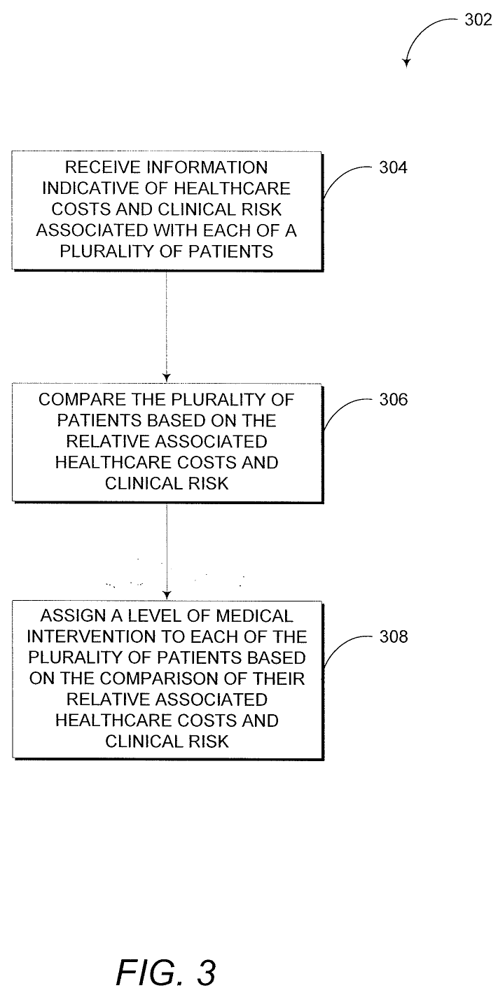

FIG. 3 is a flow chart illustrating one embodiment of a process 302 for assigning a level of medical intervention to a plurality of patients. Beginning at a state 304, the process 302 receives information indicative of healthcare costs and clinical risk associated with each of a plurality of patients. In one embodiment, the process 302 receives health information about the plurality of patients and calculates the predicted healthcare costs and clinical risk associated with each of a plurality of patients. In another embodiment, the process 302 receives predictive models scores that have already been calculated. The predictive model scores may be output from the predictive models discussed above in relation to FIG. 2, such as the high cost predictive model 206 and the clinical risk predictive model 208. In one embodiment, the process 302 receives four predictive model scores for each of the plurality of patients: a score from the end of life predictive model 202, a score from a cost pattern predictive model 204, a score from a high cost predictive model 206, and a score from a clinical risk predictive model 208.

Proceeding to a state 306, the healthcare management system 100 compares the plurality of patients based on the relative associated healthcare costs and clinical risk of each of the patients. In one embodiment, the process 302 creates a ranking index based on a high cost score received from the future high cost predictive model 206 and an adjusted risk score received from the clinical risk predictive model 208. The process 302 may then assign a rank index to each patient.

In one embodiment, the patients are compared using an x-y analysis. For example, an x-y chart may be created where the x-axis represents a high cost predictive model score and the y-axis represents a clinical risk predictive model score. The patients may be plotted along the x-y chart based on these two values, and patients may be grouped based on their position in the chart. In another embodiment, patients are ranked in a first list in order of high cost predictive model score and ranked in a second list based on a clinical risk predictive model score. Any suitable comparison method may be used.

In one embodiment, after an x-y chart is created, the patients may be group together based on their position in the chart. In one embodiment, the process 302 groups patients based on the clinical risk and high cost predictive model scores associated with each patient. In another embodiment, the process 302 receives some user input regarding how to group the patients based on the scores.

Continuing to a state 308, the process 302 assigns a level of medical intervention to each of the plurality of patients based on the comparison of their relative associated healthcare costs and clinical risk. The level of medical intervention may determine, for example, the type of care and communications that the patient should receive.

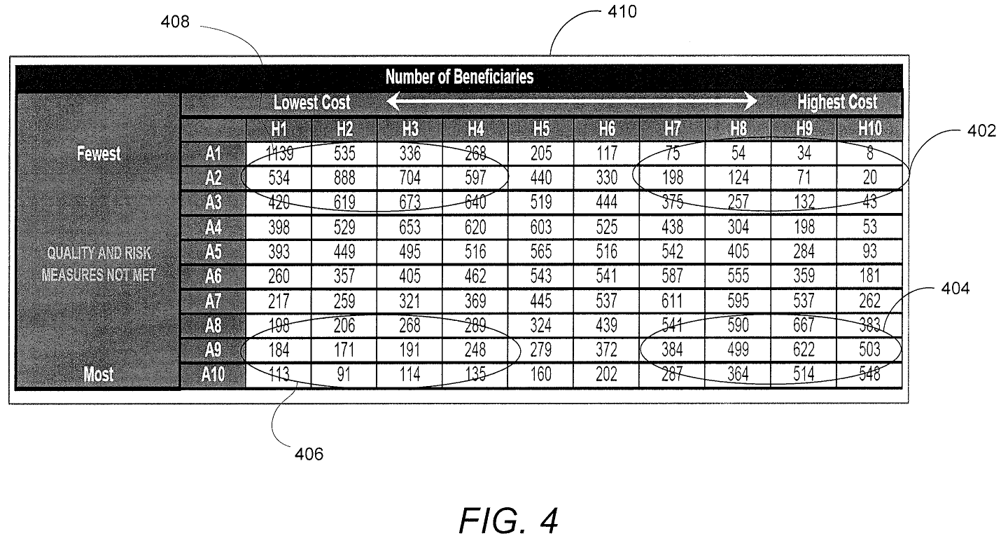

FIG. 4 is a diagram illustrating one embodiment of an example matrix 410 used for assigning patients to a level of medical intervention. FIG. 4 shows a matrix with the cost plotted on the x-axis, shown in blocks of H1-H10, and the risk plotted on the y-axis, shown in blocks A1-A10. Based on the predictive model scores, the healthcare management system 100 divides the patients into various states, or buckets, within the matrix. In one embodiment, each block in the matrix represents an individual patient, where the value in each block in the matrix represents a score indicative of a combined high cost predictive model score and a clinical risk predictive model score. In another embodiment, each block on the matrix represents a group of patients, where the value in each block represents the number of patients in that grouping, as shown in the matrix 410.

The matrix 410 may be used to analyze which patients should receive a higher level of care. For example, patient group 408 has both a predicted low value for the high cost model score (indicative of predictive low future health costs) and a low score for clinical risk, meaning that these patients have a predicted low cost and few quality and risk measures not met. There is likely little to be done for these patients at this time. This group most likely does not require a heightened level of medical intervention.

Patients in patient group 402 have a projected high cost, but few quality/risk measure not met. Because there are few quality and risk measures for which intervention is available, there is little to do to mitigate these potential high costs. It is unlikely to be desirable to assign patient group 402 to a heighted level of medical intervention.

Patient group 406 has a projected low cost and the highest number of quality/risk measures not met. Although there are quality and risk measures not being met, the prediction of low costs means that it is unlikely to be desirable to provide these patients with many medical interventions.

Patient group 404 has a projected high cost and the highest number of quality/risk measures not met. Because of this combination, the healthcare system 100 may choose these patients in patient group 404 to receive a heighted level of medical interventions. Because of the high cost prediction and increased number of quality and risk measures not being measured, the majority of interventions should be focused here in an effort to mitigate the most healthcare cost.

In one embodiment, patients with a particular score combination are added to a particular level of medical intervention. In another embodiment, a certain number or certain percentage of patients may be assigned to a particular level of medical intervention, for example when a level of healthcare intervention corresponds to a type of healthcare program with limited availability. The number of patients for a particular level of medical intervention and the individual levels of medical intervention may be variable. In one embodiment, the process 302 receives information and analyzes the information in order to determine how to set up the healthcare programs. The process 302 may move along the chart filling a particular level of medical intervention with patients until full.

### Determining the Eligible Population

In one embodiment, the healthcare management system 100 selects a subset of the plurality of patients for comparing the patients based on relative associated healthcare costs and clinical risks and assigns these patients to a level of medical intervention without the cost and risk analysis provided by the process 302 from FIG. 3. For example, the predictive model scores of some patients may indicate that the patient should automatically be in a particular intervention level and no further analysis is needed for that group of patients. The healthcare management system 100 may select the subset of the plurality of patients based on one or more predictive model scores.

The healthcare management system 100 may analyze a predictive modeling score received from the end of life predictive model 202 in order to determine whether a patient is likely to die within the near future. If the patient is likely to die in the near future, the healthcare management system 100 may assign the patient to a particular level of medical intervention designed for patients likely to be the end of their life. The healthcare management system 100 may remove these patients from the group which the healthcare management system 100 is comparing high cost and clinical risk predictive model scores to determine the level of medical intervention. In one embodiment, the healthcare management system 100 compares the predictive model scores from the end of life predictive model 202 and assigns a particular percentage or number of patients to the end of life medical intervention level based on the end of life level predictive model scores. In another embodiment, any patient with an end of life score at a particular level is assigned to the end of life level intervention.

In one embodiment, the healthcare management system 100 may remove the patients that have medical costs that are leveling off from the group eligible for intervention. In one embodiment, the healthcare management system 100 uses a predictive model score from the past cost pattern predictive model 204. The past cost pattern predictive model score may indicate whether healthcare cost claims have started to level off, or de-escalate, for a group or an individual. In one embodiment, the healthcare management system 100 determines a particular percentage of patients based on the predictive model score. In another embodiment, a particular number or range of numbers of patients is chosen. In one embodiment, patients with stabilizing medical costs have limited eligibility for assignment to a heightened medical intervention group. In one embodiment, patients identified as being near the end or life or having stabilizing medical costs are excluded from the remaining analysis, such as the risk versus cost analysis.

From this initial target group containing patients not found to be near the end of life and not having stabilizing medical costs, the additional predictive models are run that are relevant for a particular program offering. In one embodiment, these models are run according to the process 302 of FIG. 3. The result is a smaller target group that, based on the results of the models, is predicted to have the highest likelihood for increased health costs. This smaller target group receives the most focus under the program offering.

In one embodiment, other factors are also used to determine which patients are eligible for particular levels of medical intervention and programs within levels of medical intervention. In some embodiments, the members that may be eligible for the program may be disease agnostic.

In some embodiments, information about the healthcare coverage of the patient is also used to determine which patients should receive further analysis in order to determine if they are eligible for heightened medical interventions. In certain embodiments, members must have current active coverage to be eligible to participate. In certain embodiments, members who have chosen to opt out of one program assignment are no longer eligible for heightened medical intervention or participation in that same program type. In one embodiment, members who have been clinically opted out of a medical program may later be eligible for the program again. If the patient opted out of a program due to a decision by a healthcare provider or a clinician, the patient may enter back into that program at a later date. In one embodiment, a patient may opt out of only a subset of programs. In another embodiment, the patient must opt in or out of all heightened medical intervention programs. In some embodiments certain rules may apply only to particular clients. For example, in some embodiments, dependents and spouses of patients may not be eligible for certain programs.

### Examples of Levels of Medical Intervention

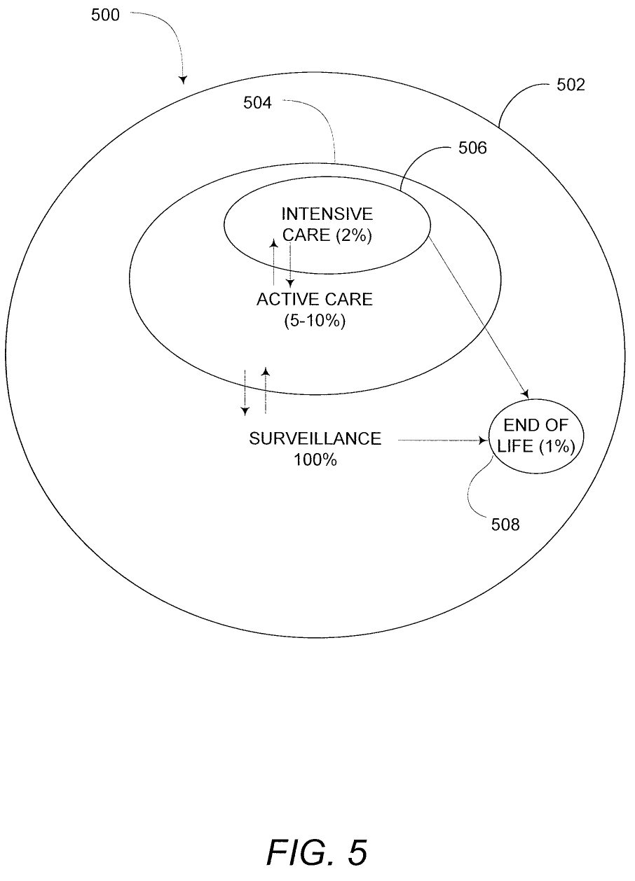

FIG. 5 is a diagram of one embodiment of a chart 500 depicting possible assignments of patients to levels of medical intervention. In one embodiment, the levels of medical intervention include a surveillance level 502, an end of life level 508, an intensive care level 506, and an active care level 504. However, it will be appreciated that any desired number or any desired categories of intervention may be provided. The medical goals for each group may differ. For example, the goal of care for the group of patients in the surveillance level 508 may be to keep the healthy patients well. The goal for patients in the active care level 504 may be to manage identified health risks. The goal for patients in the intensive care level 506 may be to prevent an avoidable health crisis. The goal for patients in the end of life level 508 may be to provide specialized support for members and care givers. The level of medical intervention may vary based on the assigned level. In one embodiment, one percent of patients are assigned to the end of life level, two percent of patients are assigned to the intensive care level 506, five to ten percent of patients are assigned to the active care level 504, and one hundred percent of patients are assigned to the surveillance level 502. In other embodiments, other percentages or numbers of patients may be assigned to each level of medical intervention.

In some embodiments, patients assigned to the surveillance level 502 may receive reminders about preventive programs. These patients may also receive information about care transition if the patient is hospitalized. Patients only assigned to the surveillance level 502 are the lowest acuity members, and the goal is to ensure that risk factors are identified before cost and care needs escalate. Actions include, for example, processing claims monthly to ensure no new triggers, and/or sending mailings and reminders about vaccines. Patients may be upgraded into a higher level of care. In one embodiment, all patients are assigned to the surveillance level 502, and patients may be assigned to this level as well as another level.

Patients in the active care level 504 may be assigned to a specific active care management team, and they may also be targeted for specific interventions. In some embodiments, interventions for these patients may be proactive, patient-centric, and delivered on a nurse team model where members have access to multiple health practitioners, such as nurses, dieticians, and respiratory therapists. Programs may be designed to help members learn how to manage their condition or multiple-morbidities and to learn healthy living habits so they can graduate to the surveillance level 502. In some embodiments, interventions may be disease agnostic, employing a “whole person” approach to care, including for example, coaching on advanced planning, evaluations of action plan, assessment of barriers to compliance with standards of care, evaluation of fail risk, and depression screening. If desired, members may also be upgraded to the intensive care level 506 if their health deteriorates or costs increase.

In one embodiment, patients in the intensive care level 506 are also assigned to the active care level 504 and the surveillance level 502. In some embodiments, patients in the intensive care level 506 may be assigned a one to one ratio clinician patient ratio for care and may also be targeted for specific interventions. If desired, interventions for these patients may be proactive, patient-centric, and delivered on a one-to-one nurse case management tool. Programs may be designed to avoid repeated hospitalizations. Programs may include care planning and coordination, coaching and monitoring, and assessments based on the following: psycho-social interventions, motivational interviewing, hospital discharge planning and post-hospital discharge programs. In one embodiment programs are also designed to reinforce a physician's action plan and to avoid re-hospitalization. The programs may also notify members of available benefits and resources. Members may graduate to active care level 504 management as appropriate.

Patients assigned to the end of life level 508 of medical intervention may receive one to one management, care transition, caregiver support, and other advanced directives. This allows for the unique ability to identify end of life members for appropriate planning and intervention. Highly personalized interventions may be done to help members and control costs in the last six months, or other selected time frame, of life. Interventions may include, for example, advanced planning, assessment of comfort and symptoms management, education around initiating discussions with doctors and family, coordination with hospice, education around benefits of palliative care, and assistance with transition from curative to palliative care.

In one embodiment, other predictive models and levels of medical care may also be used. For example, there could be a special level of medical intervention for certain conditions, such as an oncology level of medical intervention.

### Example of One Embodiment of Assigning a Level of Medical Intervention

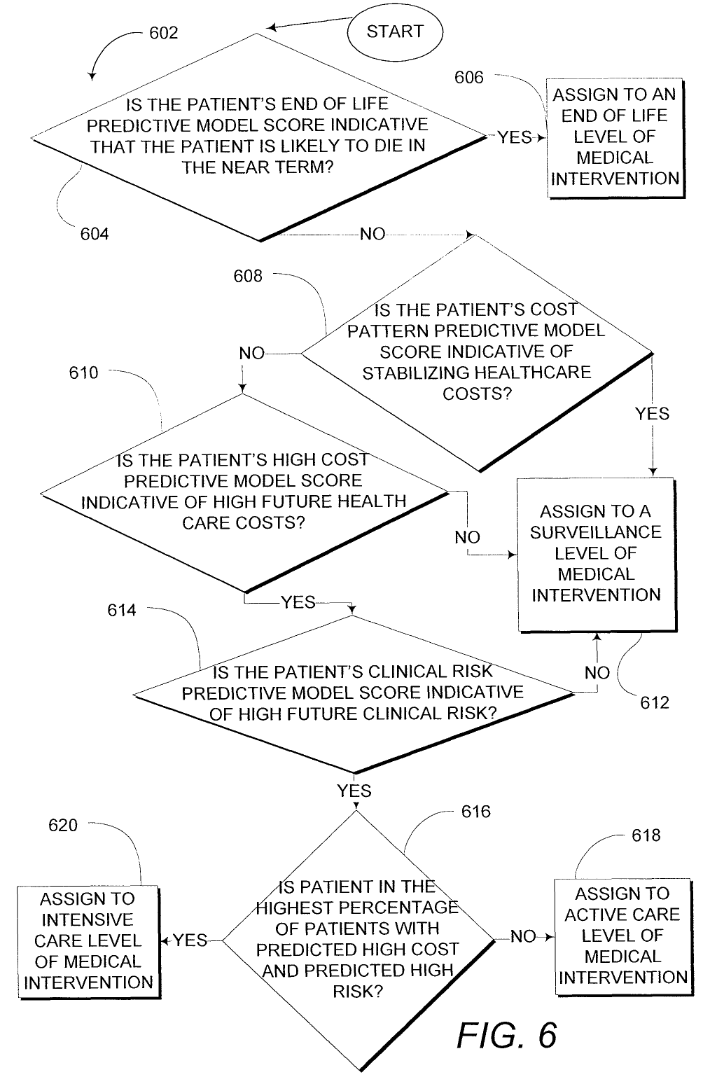

FIG. 6 is a flow chart illustrating one embodiment of a process 602 for assigning a patient to either the end of life level 508, surveillance level 502, intensive care level 506, or active care level 504 of medical intervention as shown in FIG. 5. FIG. 6 depicts one example of one embodiment of the process 302 shown in FIG. 3. Beginning at a decision state 604, the process 602 determines whether the patient's end of life predictive model score is indicative that the patient is likely to die in the near term. If so, the healthcare management system 100 moves to a state 606 and assigns the patient to the end of life level 508 of medical intervention.

If the patient's end of life predictive model score is not indicative of a high likelihood of death in the near term, the process 602 moves to a decision state 608 and determines whether the patient's cost pattern predictive model score is indicative of stabilizing healthcare costs. If so, the process 602 advances to a state 612 and assigns the patients to the surveillance level 502 of medical intervention.

If the patient's cost pattern predictive model score is not indicative of stabilizing healthcare costs, the process 602 moves to a decision state 610 to determine whether the patient's high cost predictive model score is indicative of future high healthcare costs. If not, the process 602 proceeds to a state 612 and assigns the patient to the surveillance level 502.

If the patient's high cost predictive model score is indicative of future high healthcare costs, the process 602 advances to a decision state 614 and determines whether the patient's clinical risk predictive model score is indicative of high future clinical risk. If not, the process 602 again advances to the state 612 and assigns the patient to the surveillance level 502. If the patient's high cost predictive model score is indicative of high costs and the patient's clinical risk predictive model score is indicative of high clinical risk, the process 602 determines whether to assign the patient to the active care level 504 or intensive care level 506.

The process 602 moves to a decision state 616 and determines whether the patient is in the highest percentage of patients with predicted high cost and predicted high clinical risk. If not, the process 602 assigns the patient to the active care level of medical intervention at state 618. However, if the patient is in the set of patients with the highest combination of high cost predicted clinical risk, process 602 assigns the patient to intensive care level of medical intervention at state 620.

It will be appreciated that the processes and decision states in the method described above may be performed in any desired order and need not be performed in the order set forth above.

### Updating Patient Levels of Medical Intervention

In one embodiment, the healthcare management system 100, such as through process 302 or 602, dynamically assigns patients to levels of medical intervention. The healthcare management system 100 may re-assign patients on a regular basis in order to adjust the model to changing patient health statuses. This allows the healthcare management system 100 to identify and assign patients at addressable junctures rather than waiting until the situation is irreversible. In one embodiment, the healthcare management system 100 re-evaluates patients on a monthly basis, for example, to realign the level of medical intervention. In one embodiment, the frequency of the update is stored in a database.

In one embodiment, the reevaluation process involves ensuring that the percentage or number of patients in each level of medical intervention is the desired number or percentage of patients. In one embodiment, the allowable number of patients in each level of medical intervention may also be dynamic. In one embodiment, the number or percentage of patients may only vary by a particular amount. The movement of the patients among levels of medical intervention and the number and identification of patients in each level of medical intervention may be stored in a database. In one embodiment, the healthcare management system 100 determines and adjusts the eligible population for heightened medical intervention at regular intervals. The healthcare management system 100 may also re-assign patients to programs within a level of medical intervention at regular intervals.

Those of skill in the art will recognize that the various illustrative logical states, modules, circuits, and algorithm steps described in connection with the embodiments disclosed herein may be implemented as electronic hardware, computer software, or combinations of both. To clearly illustrate this interchangeability of hardware and software, various illustrative components, states, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical states, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

While the above detailed description has shown, described, and pointed out novel features of the invention as applied to various embodiments, it will be understood that various omissions, substitutions, and changes in the form and details of the device or process illustrated may be made by those skilled in the art without departing from the intent of the invention. As will be recognized, the present invention may be embodied within a form that does not provide all of the features and benefits set forth herein, as some features may be used or practiced separately from others. The scope of the invention is indicated by the appended claims rather than by the foregoing description. All changes which come within the meaning and range of equivalency of the claims are to be embraced within their scope.

What is claimed is:

1. A computerized method for targeting a group of patients for medical care, the method comprising:
receiving health information about a plurality of patients;
selecting a subset of the plurality of patients;
comparing the relative associated future healthcare costs and clinical risk of the subset of the plurality of patients; and
assigning a level of medical intervention for each of the patients in the selected subset based on the comparison of the patients.

2. The method of claim 1, wherein the received health information comprises healthcare insurance claims information.

3. The method of claim 1, further comprising creating a medical care plan based on the assigned level of medical intervention.

4. The method of claim 1, wherein selecting a plurality of patients comprises selecting a plurality of patients based on the likelihood of each patient dying within a particular time range.

5. The method of claim 1, wherein selecting a plurality of patients comprises selecting a plurality of patients based on the likelihood of the future healthcare costs for each patient stabilizing.

6. The method of claim 1, wherein assigning a level of medical intervention comprises assigning patients with high future healthcare costs and high clinical risk to an increased level of medical intervention.

7. The method of claim 1, further comprising periodically reassigning patients to a different level of medical intervention.

8. The method of claim 1, further comprising assigning the non-selected patients to a level of medical intervention.

9. The method of claim 1, wherein a computing device assigns the level of medical intervention.

10. The method of claim 1, further comprising delivering the assigned level of medical intervention to a patient in the selected subset.

11. The method of claim 1, wherein selecting the subset of the plurality of patients comprises both selecting a plurality of patients based on the likelihood of each patient dying within a particular time range and selecting a plurality of patients based on the likelihood of the future healthcare costs for each patient stabilizing.

12. A computerized method for targeting a group of patients for medical care, the method comprising:
receiving health information associated with each of a plurality of patients;
receiving output from multiple predictive models about the plurality of patients;
comparing a subset of the plurality of patients relative to one another based on the output of the multiple predictive models; and
assigning a level of medical intervention for each of the patients based on the comparison of the patients.

13. The method of claim 12, wherein the received health information comprises healthcare insurance claims information.

14. The method of claim 12, further comprising creating a medical care plan based on the assigned level of medical intervention.

15. The method of claim 12, wherein a computing device assigns the level of medical intervention.

16. The method of claim 12, wherein the output from the multiple predictive models comprises output indicative of each patient's likelihood of dying in the near term.

17. The method of claim 12, wherein the output from the multiple predictive models comprises output indicative of the likelihood of each patient's healthcare costs stabilizing.

18. The method of claim 12, wherein the output from the multiple predictive models comprises output indicative of the relative level of each patient's future healthcare costs.

19. The method of claim 12, wherein the output from the multiple predictive models comprises output indicative of each patient's relative future clinical risk.

20. The method of claim 12, wherein patients with an associated predictive model output indicative of a higher relative level of future healthcare costs and a higher relative future clinical risk are assigned to an increased level of medical intervention.

21. The method of claim 12, wherein the levels of medical intervention comprise a surveillance level, an end of life level, and an intermediate level of medical intervention.

22. The method of claim 12, further comprising periodically reassigning patients to a different level of medical intervention.

23. The method of claim 12, further comprising delivering the assigned level of medical intervention to a patient based on the comparison of the patients.

24. The method of claim 12, wherein the output from the multiple predictive models comprises two or more outputs selected from the group consisting of output indicative of each patient's likelihood of dying in the near term, output indicative of the likelihood of each patient's healthcare costs stabilizing, output indicative of the relative level of each patient's future healthcare costs and output indicative of each patient's relative future clinical risk.

25. The method of claim 12, wherein the output from the multiple predictive models comprises:
output indicative of each patient's likelihood of dying in the near term;
output indicative of the likelihood of each patient's healthcare costs stabilizing;
output indicative of the relative level of each patient's future healthcare costs; and
output indicative of each patient's relative future clinical risk.

26. A computerized method for targeting a group of patients for medical care, the method comprising:
selecting a plurality of patients;
determining an estimated medical factor associated with each patient;
comparing the patients based on the associated medical factor of each patient; and
assigning a level of medical intervention for each patient based on the relative estimated medical factor of each patient.

27. The method of claim 26, wherein the estimated medical factor comprises the estimated future medical costs associated with each patient.

28. The method of claim 27, wherein the estimated medical factor further comprises the estimated future clinical risk associated with each patient.

29. The method of claim 28, wherein patients with a high estimated future medical costs and high estimated clinical risk relative to the other patients are assigned to an increased level of medical intervention.

30. The method of claim 26, wherein selecting a plurality of patients comprises selecting a plurality of patients based on the likelihood of each patient dying within a particular time range.

31. The method of claim 26, wherein selecting a plurality of patients comprises selecting a plurality of patients based on the likelihood of the future medical costs for each patient stabilizing.

32. The method of claim 26, wherein determining an estimated medical factor comprises analyzing output from one or more predictive models.

33. The method of claim 32, wherein the one or more predictive models are neural networks.

34. The method of claim 26, wherein the levels of medical intervention comprise a surveillance level and active care level of medical intervention.

35. The method of claim 26, further comprising creating a medical plan based on the assigned level of medical intervention.

36. The method of claim 26, further comprising assigning the non-selected patients to a level of medical intervention.

37. The method of claim 26, wherein a computing device assigns the level of medical intervention.

38. The method of claim 26, wherein selecting a plurality of patients comprises both selecting a plurality of patients based on the likelihood of each patient dying within a particular time range and selecting a plurality of patients based on the likelihood of the future healthcare costs for each patient stabilizing.

39. The method of claim 26, further comprising delivering the assigned level of medical intervention to a patient based on the relative estimated medical factor of said patient.

40. A computerized system for targeting a group of patients for medical care, the system comprising:
a memory to store health information about a plurality of patients;
a processor to perform:
selecting a subset of the plurality of patients;
comparing the relative associated future healthcare costs and clinical risk of the subset of the plurality of patients; and
assigning a level of medical intervention for each of the patients in the selected subset based on the comparison of the patients.

41. The system of claim 40, wherein the received health information comprises healthcare insurance claims information.

42. The system of claim 40, wherein the processor further performs creating a medical care plan based on the assigned level of medical intervention.

43. The system of claim 40, wherein selecting a plurality of patients comprises selecting a plurality of patients based on the likelihood of each patient dying within a particular time range.

44. The system of claim 40, wherein selecting a plurality of patients comprises selecting a plurality of patients based on the likelihood of the future healthcare costs for each patient stabilizing.

45. The system of claim 40, wherein assigning a level of medical intervention comprises assigning patients with high future healthcare costs and high clinical risk to an increased level of medical intervention.

46. The system of claim 40, wherein the processor further performs periodically reassigning patients to a different level of medical intervention.

47. The system of claim 40, wherein the processor further performs assigning the non-selected patients to a level of medical intervention.

48. The system of claim 40, wherein selecting the subset of the plurality of patients comprises both selecting a plurality of patients based on the likelihood of each patient dying within a particular time range and selecting a plurality of patients based on the likelihood of the future healthcare costs for each patient stabilizing.

49. A computerized system for targeting a group of patients for medical care, the system comprising:
a memory to store health information associated with each of a plurality of patients;
a processor to perform:
receiving output from multiple predictive models about the plurality of patients;
comparing a subset of the plurality of patients relative to one another based on the output of the multiple predictive models; and
assigning a level of medical intervention for each of the patients based on the comparison of the patients.

50. The system of claim 49, wherein the received health information comprises healthcare insurance claims information.

51. The system of claim 49, wherein the processor further performs creating a medical care plan based on the assigned level of medical intervention.

52. The system of claim 49, wherein the output from the multiple predictive models comprises output indicative of each patient's likelihood of dying in the near term.

53. The system of claim 49, wherein the output from the multiple predictive models comprises output indicative of the likelihood of each patient's healthcare costs stabilizing.

54. The system of claim 49, wherein the output from the multiple predictive models comprises output indicative of the relative level of each patient's future healthcare costs.

55. The method of claim 49, wherein the output from the multiple predictive models comprises output indicative of each patient's relative future clinical risk.

56. The system of claim 49, wherein patients with an associated predictive model output indicative of a higher relative level of future healthcare costs and a higher relative future clinical risk are assigned to an increased level of medical intervention.

57. The system of claim 49, wherein the levels of medical intervention comprise a surveillance level, an end of life level, and an intermediate level of medical intervention.

58. The system of claim 49, wherein the processor further performs periodically reassigning patients to a different level of medical intervention.

59. The system of claim 49, wherein the output from the multiple predictive models comprises two or more outputs selected from the group consisting of output indicative of each patient's likelihood of dying in the near term, output indicative of the likelihood of each patient's healthcare costs stabilizing, output indicative of the relative level of each patient's future healthcare costs and output indicative of each patient's relative future clinical risk.

60. The system of claim 49, wherein the output from the multiple predictive models comprises:
output indicative of each patient's likelihood of dying in the near term;
output indicative of the likelihood of each patient's healthcare costs stabilizing;
output indicative of the relative level of each patient's future healthcare costs; and
output indicative of each patient's relative future clinical risk.

61. A computerized system for targeting a group of patients for medical care, the system comprising:
a processor to perform:
selecting a plurality of patients;
determining an estimated medical factor associated with each patient;
comparing the patients based on the associated medical factor of each patient; and
assigning a level of medical intervention for each patient based on the relative estimated medical factor of each patient.

62. The system of claim 61, wherein the estimated medical factor comprises the estimated future medical costs associated with each patient.

63. The system of claim 62, wherein the estimated medical factor further comprises the estimated future clinical risk associated with each patient.

64. The system of claim 63, wherein patients with a high estimated future medical costs and high estimated clinical risk relative to the other patients are assigned to an increased level of medical intervention.

65. The system of claim 63, wherein selecting a plurality of patients comprises selecting a plurality of patients based on the likelihood of each patient dying within a particular time range.

66. The system of claim 61, wherein selecting a plurality of patients comprises selecting a plurality of patients based on the likelihood of the future medical costs for each patient stabilizing.

67. The system of claim 61, wherein determining an estimated medical factor comprises analyzing output from one or more predictive models.

68. The system of claim 67, wherein the one or more predictive models are neural networks.

69. The system of claim 61, wherein the levels of medical intervention comprise a surveillance level and active care level of medical intervention.

70. The system of claim 61, wherein the processor further performs creating a medical plan based on the assigned level of medical intervention.

71. The system of claim 61, wherein the processor further performs assigning the non-selected patients to a level of medical intervention.

72. The system of claim 61, wherein selecting a plurality of patients comprises both selecting a plurality of patients based on the likelihood of each patient dying within a particular time range and selecting a plurality of patients based on the likelihood of the future healthcare costs for each patient stabilizing.
